(19) United States
(12) Patent Application Publication
(10) Pub. No.: US 2024/0018452 A1
(43) Pub. Date: Jan. 18, 2024

(54) CHIP FOR INTEGRATED TUMOR CELL BEHAVIOR EXPERIMENTS

(71) Applicants: Zhiyuan LI, Guangzhou (CN); Rongqi HUANG, Guangzhou (CN); Shuai LI, Guangzhou (CN); Chao TIAN, Guangzhou (CN); Zuoxian LIN, Guangzhou (CN); Na CHENG, Guangzhou (CN)

(72) Inventors: Zhiyuan LI, Guangzhou (CN); Rongqi HUANG, Guangzhou (CN); Shuai LI, Guangzhou (CN); Chao TIAN, Guangzhou (CN); Zuoxian LIN, Guangzhou (CN); Na CHENG, Guangzhou (CN)

(21) Appl. No.: 18/026,325

(22) PCT Filed: Dec. 30, 2020
(86) PCT No.: PCT/CN2020/141199

§ 371 Date: Mar. 14, 2023

(30) Foreign Application Priority Data
Dec. 25, 2020 (CN) 202011564189.9

(51) Int. Cl. C12M 3/00; C12M 1/00

(52) U.S. Cl. CPC C12M 21/08; C12M 23/34; C12N 2513/00

(57) ABSTRACT

An chip for integrated tumor cell behavior experiments, which comprises a functional area I, a functional area II, a functional area III, a functional area IV and a functional area V, wherein the functional area I comprises a cell invasion 3D co-culture plate (400) for cell invasion experiments; the functional area II comprises a cell migration culture hole (500) for cell migration experiments; the functional area III comprises a cell proliferation single-cell culture hole (600) for tumor single-cell culture; the functional area IV comprises an angiogenesis 3D co-culture plate (700) for tumor-related angiogenesis experiments; and the functional area V comprises a tumor single-cell culture hole (803), a matrix glue groove (805) and a tumor cell attraction factor hole (801) connected by matrix glue for tumor single-cell migration or invasion experiments. The single-cell culture, micro-fluidic and 3D culture techniques are comprehensively used in the chip, such that the experiment process is obviously simplified, the experiment efficiency is improved, and the obtained experiment result has a higher accuracy and repeatability.

### TECHNICAL FIELD

The present application relates to the field of tumor cell biology and, in particular, to an integrated cell culture chip, especially an integrated chip for tumor cell behavior experiments.

### BACKGROUND

To culture tumor cells in vitro and study tumor cell behaviors such as the proliferation, migration and invasion of tumor cells as well as the effect of tumor cells on angiogenesis is not only an important link in researches in tumor cell biology but also an important basis to explore the cause and development mechanism of tumors and screen anti-tumor drugs. The malignant behaviors of tumors are embodied in the infinite proliferation, migration and invasion of tumor cells and the promotion of neovascularization by tumor cells. Therefore, almost all the above related cell experiments need to be conducted during the investigation of tumor cell behaviors.

At present, cell proliferation experiments include an MTT method and a CCK-8 method, both of which rely on a colorimetric method to indirectly measure cell proliferation indicators and can estimate the proliferation situation of a tumor cell population. However, the MTT method has the disadvantages of a reagent insoluble in water, susceptibility to serum and drugs, a need to add the reagent without light, etc. and the CCK-8 method is relatively complicated to operate and high in cost. Additionally, neither of these two methods can investigate the proliferation situation of a single cell.

Common cell migration experiments include a cell scratch method or a Transwell method, where the scratch method needs to use a pipette head for scratching and its operation repeatability is not stable enough, while the Transwell method has the disadvantages of a relatively complicated operation and a difficulty in collecting data. At present, most tumor cell invasion experiments are conducted by the Transwell method. Although the invasion situation of tumor cells can be observed and measured by this method, this method is relatively complicated to operate and is not high enough in efficiency, cells need to be stained, the result is susceptible to bottom particles, and the accuracy of the method is not stable enough.

As for an experiment on the effect of tumor cells on angiogenesis, currently human umbilical vein endothelial cells (HUVECs) are often cultured using a tumor cell culture supernatant, so as to observe a neovascularization situation. This method can only observe angiogenesis but cannot investigate the direct relationship between tumor cells and the angiogenesis.

To conclude, all the above experimental methods need to be independently carried out for many times. The experiments are relatively complicated to operate, time-consuming and effort-consuming, and low in efficiency, and have the disadvantages of a difficulty in ensuring the uniformity of multiple experiments, inconvenience to observe an experimental process, a difficulty in collecting experimental data, a poor correlation between in vivo processes simulated in the experimental process, unstable accuracy, and poor experimental repeatability.

Therefore, it is significant for the study of tumor cell behaviors to provide a novel experimental chip which can simplify an experimental operation and improve experimental efficiency and has high accuracy and good repeatability.

### SUMMARY

The present application provides an integrated chip for tumor cell behavior experiments. The integrated chip for tumor cell behavior experiments comprehensively applies single-cell culture, microfluidic control and 3D culture technologies, obviously simplifies an experimental process, and significantly improves experimental efficiency, and the obtained experimental results have higher accuracy.

In a first aspect, the present application provides an integrated chip for tumor cell behavior experiments, which includes a functional area I, a functional area II, a functional area III, a functional area IV and a functional area V;
wherein, the functional area I includes cell invasion three-dimensional (3D) co-culture plates for a cell invasion experiment; the functional area II includes cell migration culture wells for a cell migration experiment; the functional area III includes cell proliferation single-cell culture wells for tumor single-cell culture; the functional area IV includes angiogenesis 3D co-culture plates for a tumor-associated angiogenesis experiment; and the functional area V includes a tumor single-cell culture well, a matrigel groove and a tumor cell attracting factor well connected by matrigel for a tumor single-cell migration or invasion experiment.

In the present application, the novel integrated cell culture chip can simultaneously conduct multiple cell behavior experiments of tumor cells once, including tumor cell behaviors such as the proliferation, migration and invasion of tumor cells and the effect of tumor cells on angiogenesis. The malignant behavior characteristics of tumor cells can be systematically investigated in one experimental cycle, which can significantly simplify an experimental process, greatly reduce experimental time and energy, improve experimental efficiency, and facilitate observation, achieves the collection of data once, easily improves the uniformity, accuracy and repeatability of experiments, and quantitatively improves the quality of the tumor cell behavior experiments.

As a preferred technical solution of the present application, the functional area I is disposed on the upper left of the integrated chip for tumor cell behavior experiments.

Preferably, the cell invasion 3D co-culture plates are in a 3×2 arrangement.

Preferably, each of the cell invasion 3D co-culture plates includes a tumor cell culture channel, a matrigel channel and a tumor cell attracting factor channel, which are next to each other in sequence.

Preferably, the tumor cell culture channel has a width of 1.5-2 mm (which may be, for example, 1.5 mm, 1.6 mm, 1.7 mm, 1.8 mm, 1.9 mm or 2 mm, etc.) and a length of 12-18 mm (which may be, for example, 12 mm, 13 mm, 14 mm, 15 mm, 16 mm, 17 mm or 18 mm, etc.).

Preferably, the matrigel channel has a width of 5-6 mm (which may be, for example, 5 mm, 6 mm, 7 mm, 8 mm, 9 mm or 10 mm, etc.) and a length of 12-18 mm (which may be, for example, 12 mm, 13 mm, 14 mm, 15 mm, 16 mm, 17 mm or 18 mm, etc.).

Preferably, the tumor cell attracting factor channel has a width of 1.5-2 mm (which may be, for example, 1.5 mm, 1.6 mm, 1.7 mm, 1.8 mm, 1.9 mm or 2 mm, etc.) and a length of 12-18 mm (which may be, for example, 12 mm, 13 mm, 14 mm, 15 mm, 16 mm, 17 mm or 18 mm, etc.).

In the present application, the invasion behavior of tumor cells is investigated by a microfluidic method in conjunction with 3D culture technology so that the observation of a tumor cell invasion process is visualized, the experiment is simpler and more accurate, more comprehensive data is obtained, and the real invasion situation of tumor cells in a human body can be better simulated.

As a preferred technical solution of the present application, the functional area II is disposed on the lower left of the integrated chip for tumor cell behavior experiments.

Preferably, the cell migration culture wells are in a 3×2 arrangement.

Preferably, a transverse septum is disposed in the middle of each of the cell migration culture wells.

In the present application, the transverse septum is a water-soluble gel material with good biological compatibility and the use of the transverse septum in the cell migration experiment can solve defects such as poor uniformity and poor stability among multiple experiments, and the operation is more convenient.

Preferably, each of the cell migration culture wells has a diameter of 8-10 mm (which may be, for example, 8 mm, 8.2 mm, 8.5 mm, 8.8 mm, 9 mm, 9.2 mm, 9.5 mm, 9.8 mm or 10 mm, etc.).

Preferably, the transverse septum has a width of 3-4 mm (which may be, for example, 3 mm, 3.2 mm, 3.5 mm, 3.6 mm, 3.8 mm or 4 mm, etc.).

Preferably, the transverse septum has a length of 8-10 mm (which may be, for example, 8 mm, 8.2 mm, 8.5 mm, 8.8 mm, 9 mm, 9.2 mm, 9.5 mm, 9.8 mm or 10 mm, etc.).

As a preferred technical solution of the present application, the functional area III is disposed in the middle of the integrated chip for tumor cell behavior experiments.

Preferably, the cell proliferation single-cell culture wells are in a 6×3 arrangement.

Preferably, each of the cell proliferation single-cell culture wells has a diameter of 7.5-8.5 mm (which may be, for example, 7.5 mm, 7.6 mm, 7.8 mm, 8 mm, 8.2 mm, 8.4 mm or 8.5 mm, etc.).

As a preferred technical solution of the present application, the functional area IV is disposed on the upper right of the integrated chip for tumor cell behavior experiments.

Preferably, the angiogenesis 3D co-culture plates are in a 3×2 arrangement.

Preferably, each of the angiogenesis 3D co-culture plates includes a tumor cell culture channel, a matrigel channel and a vascular endothelial cell culture channel, which are next to each other in sequence.

Preferably, the tumor cell culture channel has a width of 1.5-2 mm (which may be, for example, 1.5 mm, 1.6 mm, 1.7 mm, 1.8 mm, 1.9 mm or 2 mm, etc.) and a length of 12-18 mm (which may be, for example, 12 mm, 13 mm, 14 mm, 15 mm, 16 mm, 17 mm or 18 mm, etc.).

Preferably, the matrigel channel has a width of 5-6 mm (which may be, for example, 5 mm, 6 mm, 7 mm, 8 mm, 9 mm or 10 mm, etc.) and a length of 12-18 mm (which may be, for example, 12 mm, 13 mm, 14 mm, 15 mm, 16 mm, 17 mm or 18 mm, etc.).

Preferably, the vascular endothelial cell culture channel has a width of 1.5-2 mm (which may be, for example, 1.5 mm, 1.6 mm, 1.7 mm, 1.8 mm, 1.9 mm or 2 mm, etc.) and a length of 12-18 mm (which may be, for example, 12 mm, 13 mm, 14 mm, 15 mm, 16 mm, 17 mm or 18 mm, etc.).

In the present application, the neovascularization promotion process of tumor cells is investigated by 3D co-culture technology. The experimental process is more intuitive and easier to observe, the relationship between tumor cells and new microvessels can be observed directly, and the process of tumor-induced neovascularization in the human body can be better simulated.

As a preferred technical solution of the present application, the functional area V is disposed on the lower right of the integrated chip for tumor cell behavior experiments.

Preferably, one tumor single-cell culture well, one matrigel groove and one tumor cell attracting factor well on the same straight line are one culture module, six culture units are arranged radially to form one experimental unit, and experimental units in the functional area V are in a 3×2 arrangement.

In the present application, the proliferation process of tumor cells is investigated through single-cell culture, which is more convenient, accurate and personalized than a traditional experimental method.

As a preferred technical solution of the present application, the integrated chip for tumor cell behavior experiments has a length of 100-150 mm, which may be, for example, 100 mm, 110 mm, 120 mm, 130 mm, 140 mm or 150 mm, etc.

Preferably, the integrated chip for tumor cell behavior experiments has a width of 60-100 mm, which may be, for example, 60 mm, 70 mm, 80 mm, 85 mm, 90 mm or 100 mm, etc.

Preferably, the integrated chip for tumor cell behavior experiments further includes an outer cover and a base.

For example, the integrated chip for tumor cell behavior experiments includes the following five functional sites:
1. an upper left functional area includes three rows and two columns of chips with uniform specifications, each chip including channels on both sides and a matrigel channel in the middle and being used for a tumor cell invasion experiment;
2. a lower left functional area includes three rows and two columns of culture wells with uniform specifications for a cell migration experiment, where a transverse septum made of biocompatible water-soluble gel is attached to the middle of each well;
3. a middle functional area includes six rows and three columns of single-cell culture wells with uniform specifications;
4. an upper right functional area includes three rows and two columns of chips with uniform specifications, each chip including channels on both sides and a matrigel channel in the middle and being used for a tumor-promoted angiogenesis experiment; and
5. a lower right functional area includes three rows and two columns of channel areas with uniform specifications for a single-cell migration or invasion experiment.

In a second aspect, the present application provides a method for preparing the integrated chip for tumor cell behavior experiments in the first aspect, which includes:
cast-molding the integrated chip for tumor cell behavior experiments from a polymer material or integrally molding the integrated chip for tumor cell behavior experiments in a 3D printing manner.

Preferably, the polymer material includes polystyrene.

In the present application, a mold is manufactured according to technical drawings and specifications, and then the chip is cast-molded from polystyrene or integrally molded in the 3D printing manner. The entire culture chip includes an integrated base of the culture chip, one outer cover and the chip.

In a third aspect, the present application further provides a method for using the integrated chip for tumor cell behavior experiments in the first aspect. The method includes the steps below:
culturing tumor cells in functional areas with corresponding experimental functions and observing, counting or capturing pictures under a microscope to investigate the malignant behaviors of the tumor cells, including proliferation, migration, invasion and angiogenesis promotion.

In a fourth aspect, the present application further provides a use of the integrated chip for tumor cell behavior experiments in the first aspect in the study of tumor cell behaviors.

Any numerical range described in the present application includes not only the above-listed point values but also any point values within the numerical range which are not listed. Due to the limitation of space and the consideration of simplicity, specific point values included in the range are not exhaustively listed in the present application.

Compared with the existing art, the present application has at least the beneficial effects described below.

(1) The integrated chip for tumor cell behavior experiments in the present application comprehensively applies single-cell culture, microfluidic control and 3D culture technologies and can simultaneously conduct multiple cell behavior experiments of tumor cells once, including tumor cell behaviors such as the proliferation, migration and invasion of tumor cells and the effect of tumor cells on angiogenesis. The malignant behavior characteristics of tumor cells can be systematically investigated in one experimental cycle, which can simplify an experimental process, greatly reduce experimental time and energy, improve experimental efficiency, facilitate observation and achieve the collection of data once. The integrated chip for tumor cell behavior experiments provides higher accuracy for the tumor cell behavior experiments and more truly simulates the behavior patterns of in vivo tumor cells, providing a novel integrated experimental platform for exploring the cause and development mechanism of tumors and screening anti-tumor drugs.

(2) The integrated chip for tumor cell behavior experiments in the present application efficiently completes the tumor cell behavior experiments once, which include investigating the proliferation process of tumor cells through single-cell culture, investigating the invasion behavior of tumor cells (including single tumor cells) by a microfluidic method in conjunction with 3D culture technology, and investigating the neovascularization promotion process of tumor cells by 3D co-culture technology with a water-soluble gel material with good biological compatibility as the transverse septum in the cell migration experiment. The integrated experimental chip can well complete the tumor cell behavior experiments, it is prepared by a simple method, and its usage process is not complicated. The proliferation, migration and invasion processes of tumor cells can be visualized using the integrated experimental chip which is significant to popularize in the study of tumor cell behaviors.

### BRIEF DESCRIPTION OF DRAWINGS

FIG. 1 is an overall schematic diagram of an integrated chip for tumor cell behavior experiments according to the present application.

FIG. 2 is a schematic diagram of a cell invasion 3D co-culture plate.

FIG. 3 is a schematic diagram of a cell migration culture well.

FIG. 4 is a schematic diagram of a cell proliferation single-cell culture well.

FIG. 5 is a schematic diagram of an angiogenesis 3D co-culture plate.

FIG. 6 is a schematic diagram of a single-cell migration or invasion culture area.

### REFERENCE LIST

100 outer cover of a culture chip
200 base of the culture chip
300 empty groove of the culture chip
400 chip for a cell invasion experiment
401 side channel I of the chip for the cell invasion experiment
402 side channel II of the chip for the cell invasion experiment
403 matrigel channel of the chip for the cell invasion experiment
404 tumor cells invading matrigel
500 circular well for a cell migration experiment
501 transverse septum for the cell invasion experiment
502 cell spreading area for the cell migration experiment
503 tumor cells spread on both sides of the transverse septum at the beginning
504 initial septum area
505 tumor cells in migration
506 septum area gradually narrowed due to cell migration
600 cell proliferation single-cell culture well
601 spread single tumor cells
602 tumor cells proliferating initially
603 tumor cells proliferating for a period of time
700 culture chip for a tumor-associated angiogenesis experiment
701 tumor cell spreading channel for the tumor-associated angiogenesis experiment
702 matrigel channel for the tumor-associated angiogenesis experiment
703 HUVEC spreading channel for the tumor-associated angiogenesis experiment
704 new vessels generated by being induced by tumor cells
801 cell factor well for a single-cell migration/invasion experiment
802 matrigel area for the single-cell migration/invasion experiment
803 cell well for the single-cell migration/invasion experiment
804 tumor cells during migration
805 matrigel groove

### DETAILED DESCRIPTION

Technical solutions of the present application are further described below through embodiments in conjunction with drawings. However, the following examples are only simple examples of the present application and do not represent or limit the protection scope of the present application. The protection scope of the present application is subject to the claims.

In the following examples, unless otherwise specified, the experimental reagents and consumables used are purchased from conventional experiment manufacturers in the art, and the experimental methods and technical means used are conventional experimental methods and technical means known to those skilled in the art.

### Example 1

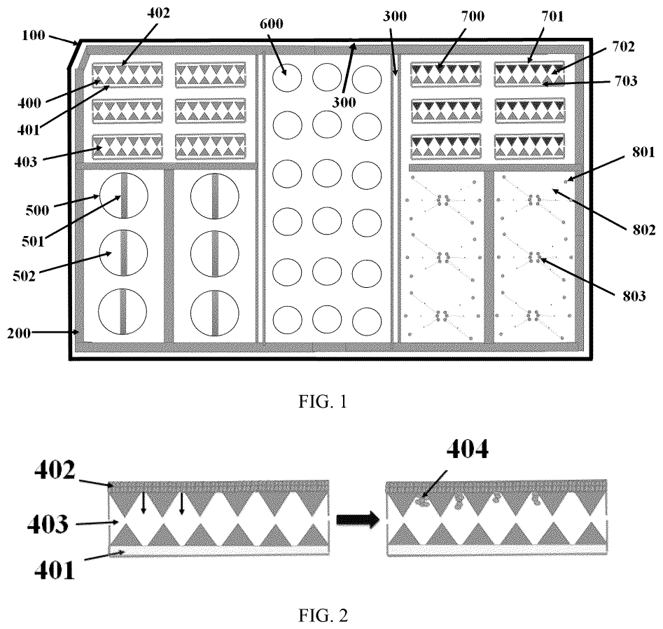

In this example, an integrated chip for tumor cell behavior experiments is provided, which has a specific structure shown in FIG. 1. The chip includes an outer cover 100 of a culture chip, a base 200 of the culture chip and empty grooves 300 of the culture chip.

Additionally, the chip further includes five functional sites which specifically include the following:

1. Upper Left Functional Area

The area includes three rows and two columns of cell invasion 3D co-culture plates with uniform specifications, that is, chips 400 for a cell invasion experiment, where each chip 400 for the cell invasion experiment includes a side channel I 401 of the chip for the cell invasion experiment and a side channel II 402 of the chip for the cell invasion experiment on both sides and a matrigel channel 403 of the chip for the cell invasion experiment in the middle.

The channels on both sides of the chip for the cell invasion experiment each have a width of 2 mm and a length of 15 mm, and the matrigel channel in the middle has a width of 5.5 mm and a length of 15 mm, where all the channels are overmolded.

2. Lower Left Functional Area

The area includes three rows and two columns of culture wells with uniform specifications for a cell migration experiment, that is, circular wells 500 for the cell migration experiment, where a transverse septum 501 for the cell invasion experiment and made of biocompatible water-soluble gel is attached to the middle of each well, and cell spreading areas 502 for the cell migration experiment are located on both sides of the transverse septum;
wherein, the circular well 500 for the cell migration experiment has a radius of 9 mm, and the transverse septum 501 for the cell invasion experiment has a width of 3.5 mm and a length of 9 mm.

3. Middle Functional Area

The area includes six rows and three columns of cell proliferation single-cell culture wells 600 with uniform specifications, where each well has a radius of 8 mm.

4. Upper Right Functional Area

The area includes three rows and two columns of angiogenesis 3D co-culture plates with uniform specifications, that is, culture chips 700 for a tumor-associated angiogenesis experiment, where each culture chip 700 for the tumor-associated angiogenesis experiment includes a tumor cell spreading channel 701 for the tumor-associated angiogenesis experiment, an HUVEC spreading channel 703 for the tumor-associated angiogenesis experiment and a middle matrigel channel 702 for the tumor-associated angiogenesis experiment;
wherein, the channels on both sides of the culture chip 700 for the tumor-associated angiogenesis experiment each have a width of 2 mm and a length of 15 mm, and the matrigel channel in the middle has a width of 5.5 mm and a length of 15 mm, where all the channels are overmolded.

6. Lower Right Functional Area

The area includes three rows and two columns of experimental units with uniform specifications for a single-cell migration/invasion experiment;
one cell factor well 801 for the single-cell migration/invasion experiment, one matrigel area 802 for the single-cell migration/invasion experiment and one cell well 803 for the single-cell migration/invasion experiment on the same straight line are one culture module, all of which are overmolded;
wherein, the cell well and the cell factor well have the same size, each well has a length of 7 mm and a width of 1.5 mm. and six culture units are arranged radially to form one experimental unit.

### Example 2

In this example, a method for using an integrated chip for tumor cell behavior experiments is provided. The method specifically includes the steps below.

1. Cell Invasion Experiment (Upper Left Functional Area)

Before the experiment, a matrigel stock solution (12 μL) was perfused with a pre-cooled small pipette into a matrigel channel (where all experimental articles need to be pre-cooled and the whole operation was performed on ice), and then a culture chip perfused with matrigel was placed in an incubator of 37° C. and stood still for 30 min to solidify the matrigel.

As shown in FIG. 2, during the experiment, the prepared (counted) tumor cell suspension (30 μL) was perfused with a medium pipette head into a side channel II 402 of the chip for the cell invasion experiment, and then a tumor cell attracting factor solution (30 μL) was perfused with a medium pipette into a side channel I 401 of the chip for the cell invasion experiment.

Next, the chip was observed under a microscope every day. Tumor cells 404 invading the matrigel can be observed in a matrigel channel 403 of the chip for the cell invasion experiment. The tumor cells 404 were counted, and captured pictures for storage, so as to facilitate comparison with a subsequent experimental situation.

2. Cell Migration Experiment (Lower Left Functional Area)

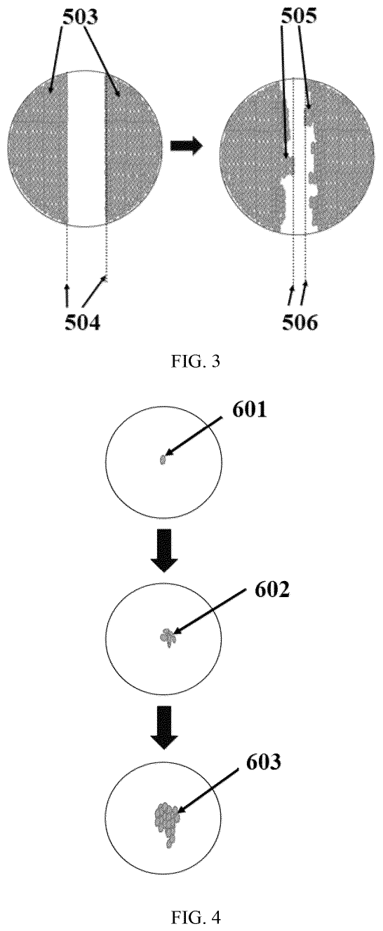

As shown in FIG. 3, before the experiment, the prepared (counted) tumor cells were spread on both sides of a transverse septum, where tumor cells 503 spread on both sides of the transverse septum at the beginning were uniformly distributed in spreading areas.

After the tumor cells adhered to walls, a culture solution was removed and then the transverse septum in each well was removed with small tweezers to leave an obvious initial septum area 504.

A culture site was observed under a microscope, and the cells on both sides were captured pictures and recorded to obtain an initial distance between the cells on both sides.

Then, the same cell culture site was observed at regular intervals to obtain the real-time migration situation of the tumor cells, where tumor cells 505 in migration and a septum area 506 gradually narrowed due to cell migration can be observed. After a certain period of culture, the closest distance between the cells on both sides was obtained and captured pictures for storage, so as to facilitate comparison with a subsequent experimental situation.

3. Tumor Single-Cell Culture (Middle Functional Area)

Before the experiment, tumor cells treated in different manners were prepared and counted.

During the experiment, every 10 μL of culture solution was diluted to a concentration of only one tumor cell with a culture medium, and the cell suspension was aspirated with a small pipette to add 10 μL of cell suspension to each culture well.

As shown in FIG. 4, the spread single tumor cell 601 gradually proliferated, and with time passing, tumor cells 602 proliferating initially and tumor cells 603 proliferating for a period of time were obtained.

After tumor cells adhered to a wall, the situation of cells in each well was observed under a microscope. A well without cells or containing more than one cell was labeled and excluded from the experiment, that is, only a culture well containing only one cell was reserved.

Next, the cell proliferation situation was observed under the microscope at regular intervals, and cells were counted and captured pictures for storage, so as to facilitate comparison with a subsequent experimental situation.

4. Tumor-Associated Angiogenesis Experiment (Upper Right Functional Area)

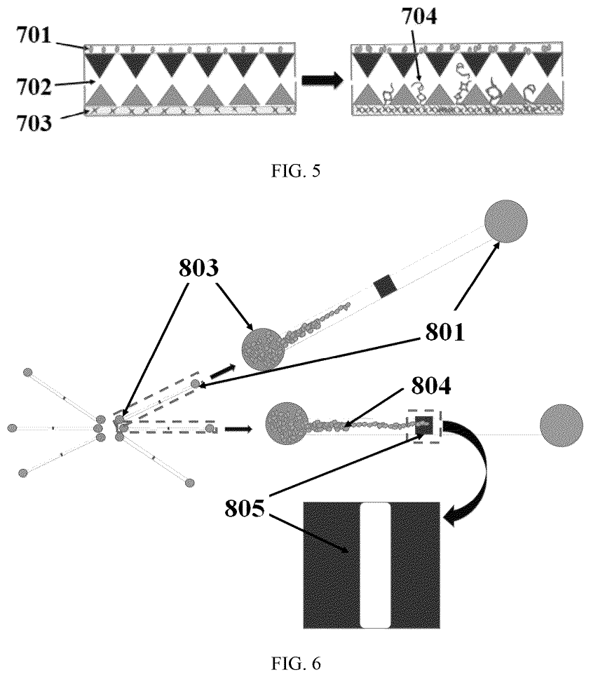

As shown in FIG. 5, before the experiment, a matrigel stock solution (12 μL) was perfused with a pre-cooled small pipette into a matrigel channel 702 for the tumor-associated angiogenesis experiment (where all experimental articles need to be pre-cooled and the whole operation was performed on ice).

Then, a culture chip perfused with matrigel was placed in an incubator of 37° C. and stood still for 30 min to solidify the matrigel.

During the experiment, the prepared (counted) tumor cells in each group were perfused into a tumor cell spreading channel 701 for the tumor-associated angiogenesis experiment, and the prepared (counted) HUVECs which had been cultured for 24 h without serum were perfused into an HUVEC spreading channel 703 for the tumor-associated angiogenesis experiment.

Next, the angiogenesis, the insertion of vessels into the matrigel and the extension of new vessels toward tumor cell wells were observed under the microscope at regular intervals. New vessels 704 generated by being induced by tumor cells can be observed. At the same time, the new vessels 704 were captured pictures, so as to facilitate comparison with a subsequent experimental situation.

5. Tumor Single-Cell Migration/Invasion Experiment (Lower Right Functional Area)

As shown in FIG. 6, two culture units (shown by two rectangular dashed boxes) and a matrigel groove 805 (shown by a square dashed box) are enlarged. Before the experiment, a matrigel stock solution (5 μL) was perfused with a pre-cooled small pipette into the matrigel groove 805 (where all experimental articles need to be pre-cooled and the whole operation was performed on ice).

Then, a culture chip perfused with matrigel was placed in an incubator of 37° C. and stood still for 30 min to solidify the matrigel.

During the experiment, the prepared tumor single-cell suspension in each group was perfused with a medium pipette into a cell well 803 for the single-cell migration/invasion experiment (35 μL per well), and the prepared attracting factor solution was perfused with the medium pipette into a cell factor well 801 for the single-cell migration/invasion experiment.

Then, the migration or invasion of cells to the matrigel was observed under the microscope at regular intervals. Tumor cells 804 during migration can be observed. The tumor cells 804 were captured pictures or counted for the subsequent statistics and comparison.

To conclude, the integrated chip for tumor cell behavior experiments in the present application comprehensively applies single-cell culture, microfluidic control and 3D culture technologies and can simultaneously conduct multiple cell behavior experiments of tumor cells once, including tumor cell behaviors such as the proliferation, migration and invasion of tumor cells and the effect of tumor cells on angiogenesis. The integrated chip greatly reduces experimental time and energy, improves experimental efficiency, facilitates observation, achieves the collection of data once, and easily improves the uniformity, accuracy and repeatability of experiments.

The applicant states that the above are only the embodiments of the present application and not intended to limit the protection scope of the present application. Those skilled in the art should understand that any changes or substitutions easily conceivable by those skilled in the art within the technical scope disclosed in the present application fall within the protection scope and the disclosed scope of the present application.

1. An integrated chip for tumor cell behavior experiments, comprising a functional area I, a functional area II, a functional area III, a functional area IV and a functional area V;
wherein, the functional area I comprises cell invasion three-dimensional (3D) co-culture plates for a cell invasion experiment;
the functional area II comprises cell migration culture wells for a cell migration experiment;
the functional area III comprises cell proliferation single-cell culture wells for tumor single-cell culture;
the functional area IV comprises angiogenesis 3D co-culture plates for a tumor-associated angiogenesis experiment; and
the functional area V comprises a tumor single-cell culture well, a matrigel groove and a tumor cell attracting factor well connected by matrigel for a tumor single-cell migration or invasion experiment.

2. The integrated chip for tumor cell behavior experiments of claim 1, wherein the functional area I is disposed on the upper left of the integrated chip for tumor cell behavior experiments.

3. The integrated chip for tumor cell behavior experiments of claim 1, wherein the cell invasion 3D co-culture plates are in a 3×2 arrangement.

4. The integrated chip for tumor cell behavior experiments of claim 1, wherein each of the cell invasion 3D co-culture plates comprises a tumor cell culture channel, a matrigel channel and a tumor cell attracting factor channel, which are next to each other in sequence;
optionally, the tumor cell culture channel has a width of 1.5-2 mm and a length of 12-18 mm;
optionally, the matrigel channel has a width of 5-6 mm and a length of 12-18 mm;
optionally, the tumor cell attracting factor channel has a width of 1.5-2 mm and a length of 12-18 mm.

5. The integrated chip for tumor cell behavior experiments of claim 1, wherein the functional area II is disposed on the lower left of the integrated chip for tumor cell behavior experiments, and the cell migration culture wells are in a 3×2 arrangement;
optionally, a transverse septum is disposed in the middle of each of the cell migration culture wells;
optionally, each of the cell migration culture wells has a diameter of 8-10 mm;
optionally, the transverse septum has a width of 3-4 mm;
optionally, the transverse septum has a length of 8-10 mm.

6. The integrated chip for tumor cell behavior experiments of claim 1, wherein the functional area III is disposed in the middle of the integrated chip for tumor cell behavior experiments;
optionally, the cell proliferation single-cell culture wells are in a 6×3 arrangement;
optionally, each of the cell proliferation single-cell culture wells has a diameter of 7.5-8.5 mm.

7. The integrated chip for tumor cell behavior experiments of claim 1, wherein the functional area IV is disposed on the upper right of the integrated chip for tumor cell behavior experiments, and the angiogenesis 3D co-culture plates are in a 3×2 arrangement;
optionally, each of the angiogenesis 3D co-culture plates comprises a tumor cell culture channel, a matrigel channel and a vascular endothelial cell culture channel, which are next to each other in sequence;
optionally, the tumor cell culture channel has a width of 1.5-2 mm and a length of 12-18 mm;
optionally, the matrigel channel has a width of 5-6 mm and a length of 12-18 mm;
optionally, the vascular endothelial cell culture channel has a width of 1.5-2 mm and a length of 12-18 mm.

8. The integrated chip for tumor cell behavior experiments of claim 1, wherein the functional area V is disposed on the lower right of the integrated chip for tumor cell behavior experiments;
optionally, one tumor single-cell culture well, one matrigel groove and one tumor cell attracting factor well on the same straight line are one culture module, six culture units are arranged radially to form one experimental unit, and experimental units in the functional area V are in a 3×2 arrangement.

9. The integrated chip for tumor cell behavior experiments of claim 1, wherein the integrated chip for tumor cell behavior experiments has a length of 100-150 mm;
optionally, the integrated chip for tumor cell behavior experiments has a width of 60-100 mm; optionally, the integrated chip for tumor cell behavior experiments further comprises an outer cover and a base.

10. A method for preparing the integrated chip for tumor cell behavior experiments of claim 1, comprising:
cast-molding the integrated chip for tumor cell behavior experiments from a polymer material or integrally molding the integrated chip for tumor cell behavior experiments in a 3D printing manner;
optionally, the polymer material comprises polystyrene.

11. A method for using the integrated chip for tumor cell behavior experiments of claim 1, comprising:
culturing tumor cells in a functional area with a corresponding experimental function and observing, counting or capturing the tumor cells under a microscope to investigate a proliferation, migration, invasion or angiogenesis promotion behavior of the tumor cells.

12. (canceled)

13. A method for studying tumor cell behaviors by using the integrated chip for tumor cell behavior experiments of claim 1.
